(19) United States
(12) Patent Application Publication
(10) Pub. No.: US 2011/0208061 A1
(43) Pub. Date: Aug. 25, 2011

(54) ULTRASONIC LESION IDENTIFICATION USING TEMPORAL PARAMETRIC CONTRAST IMAGES

(75) Inventor: Jin Chang, Mill Creek, WA (US)

(73) Assignee: KONINKLIJKE PHILIPS ELECTRONICS N.V., EINDHOVEN (NL)

(21) Appl. No.: 13/126,473

(22) PCT Filed: Oct. 27, 2009
(86) PCT No.: PCT/IB09/54751

§ 371 Date: Apr. 28, 2011

Related U.S. Application Data
(60) Provisional application No. 61/113,270, filed on Nov. 11, 2008.

(51) Int. Cl. A61B 8/14

(52) U.S. Cl. 600/458

(57) ABSTRACT

An ultrasonic diagnostic imaging system acquires a sequence of image data as a bolus of contrast agent washes into and out of a region of interest (ROI) which may contain a lesion. The image data of contrast intensity is used to compute a time-intensity curve at each point in the ROI. Levels of a time-intensity curve are set to define a rise rime period when contrast perfuses the ROI, an enhancement time period when a maximal amount of contrast is sustained in the ROI, and a fall time period when contrast washes out of the ROI. One or more of the time period parameters for the points in the ROI are used to form a parametric contrast image, which is used to identify a lesion in the ROI and its border.

This invention relates to medical diagnostic ultrasound systems and, in particular, to ultrasound systems which perform contrast-enhanced imaging studies to identify and characterize lesions such as liver tumors.

Ultrasonic contrast agents have been used for a number of years to diagnose disease states from the enhancement the agents provide to blood flow. Blood cells are very small and are poor reflectors of ultrasound, generally providing little information for ultrasonic imaging. However, microbubble contrast agents in the blood stream are highly reflective of ultrasound, enabling greatly enhanced images of blood flow characteristics. One use of contrast agents has been to identify ischemic tissue caused by a heart attack. Tissue which is ischemic and lacks blood flow will appear darker than surrounding normal myocardial tissue that is well perfused with the contrast agent. In this case it is the brightness, or signal amplitude, that is the indicator of the disease state.

Since a contrast agent can be applied in a bolus injection, and can also be disrupted by relatively intense ultrasound and allowed to reperfuse tissue, temporal characteristics of the arrival and departure of the contrast agent can also be measured and used for diagnosis. A common measure is the time-intensity curve of the arrival and departure of the contrast agent as described in U.S. Pat. No. 5,833,613 (Averkiou et al.) A time-intensity curve can be calculated for each point in an image of perfused tissue and one or more parameters of each curve for each image point can be displayed in grayscale shades or color-coding to form a parametric image of perfusion as described in U.S. Pat. No. 6,692,438 (Skyba et al.) These parameters include the peak and the slope of the curves, each indicating a different characteristic of the tissue perfusion.

A perfusion curve is generally computed by measuring the signal return from the contrast agent as it flows into and out of the microvasculature of the tissue. These measurements of the rise and fall of the amount of contrast agent are then fit to a curve such as that defined by the Gamma-variate curve model

A*(x-t₀)*exp(− [symbol] *(x-t₀))+C,
where A is the curve peak, t₀ is the time of initiation of the increase of contrast agent, [symbol] is the slope of the rise of the curve, and x is the instantaneous measurement of the amount of the contrast agent. These time and intensity representations provide an indication to a trained clinician of the manner in which the tissue is perfused.

It is known that lesions will develop their own unique microvasculature to provide a flow of blood to pathology such as cancerous lesions. Consequently the parameters of the time-intensity curve have been used to try to, first, identify a lesion and then to distinguish the lesion from surrounding normal tissue. One way this may be done is to compute and parametrically image the perfusion curve parameters of the lesion and of the normal tissue, then compare the results. Such measurements and comparisons have been used with varying results to identify and distinguish the area, shape and size of lesions. However the different parameters can give different results, and combining different parameters can yield yet a further set of results. The clinician is then put to the challenge of assessing these differing results and may have to make his own qualitative assessment of the location, size and shape of the lesion. It is desirable to more definitively locate a lesion in a contrast agent exam so that its size, shape, and particularly its border can be precisely located for subsequent treatment procedures such as hyperthermic and radiofrequency ablation therapy.

In accordance with the principles of the present invention, a diagnostic ultrasound system and method are described which enable a user to quantitatively identify and delineate a lesion and its boundary in a contrast agent exam. A perfusion curve is computed for different points in an image. Each curve is divided into parameters comprising temporal segments: the wash-in time as contrast agent perfuses the tissue location, enhancement time as the contrast agent retains it maximal amount of tissue perfusion, and wash-out time as the contrast agent washes out of the tissue location. A parametric image is formed of one or more of the temporal parameters and used to locate a lesion and, if desired, to delineate the boundary of the lesion.

In the drawings:

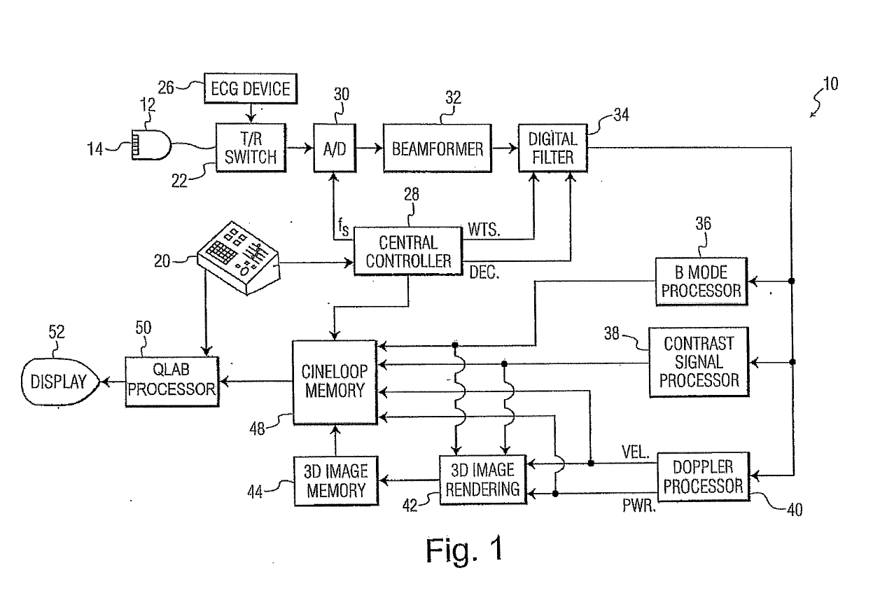

FIG. 1 illustrates in block diagram form an ultrasonic diagnostic imaging system constructed in accordance with the principles of the present invention.

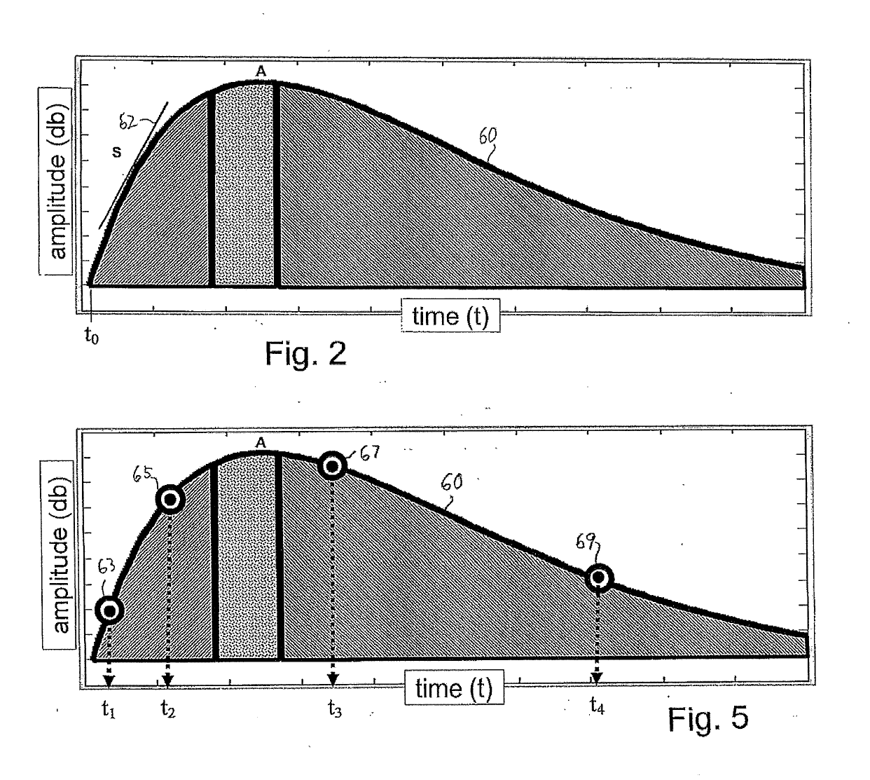

FIG. 2 illustrates a contrast agent time-intensity curve with several of the curve parameters conventionally used for contrast parametric imaging.

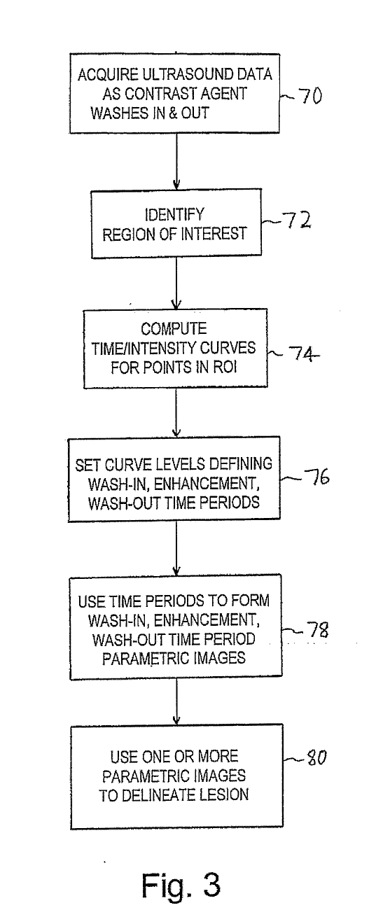

FIG. 3 is a flowchart of a process for forming a temporal contrast parametric image in accordance with the principles of the present invention.

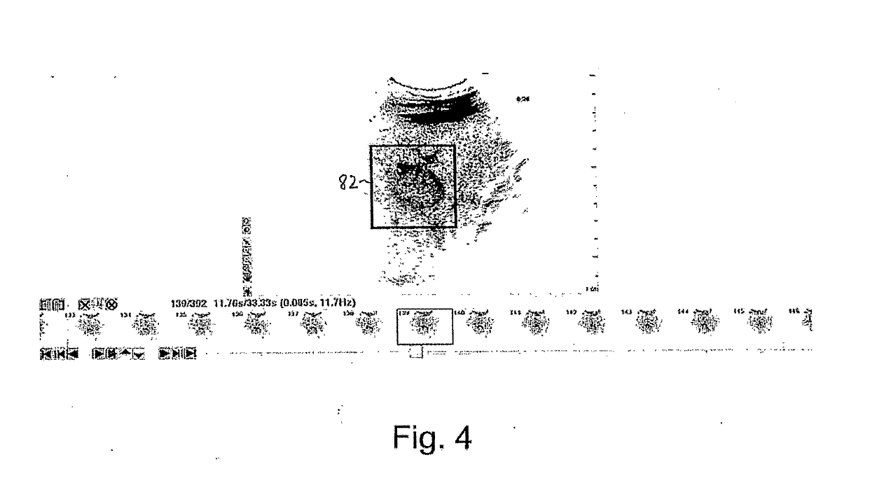

FIG. 4 illustrates a temporal contrast parametric image of the present invention which identifies the location of a lesion in a liver image.

FIG. 5 illustrates a contrast agent time-intensity curve segmented into three time periods in accordance with the present invention.

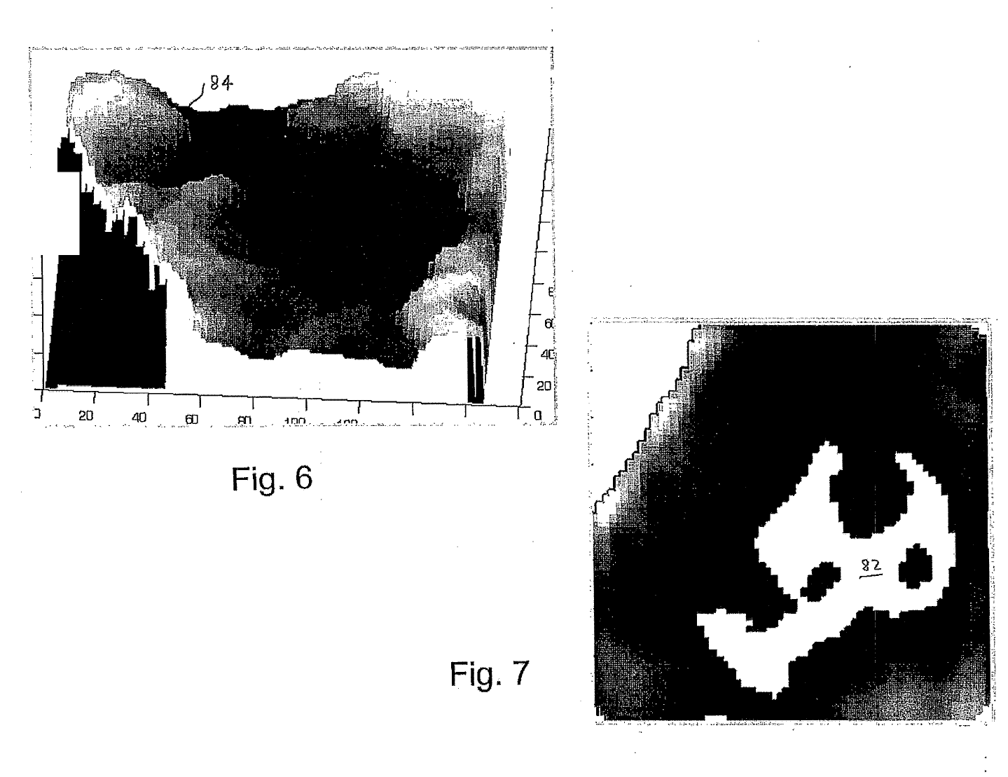

FIGS. 6 and 7 illustrate a 3D projection of a temporal contrast parametric image of the present invention which defines the border of a lesion.

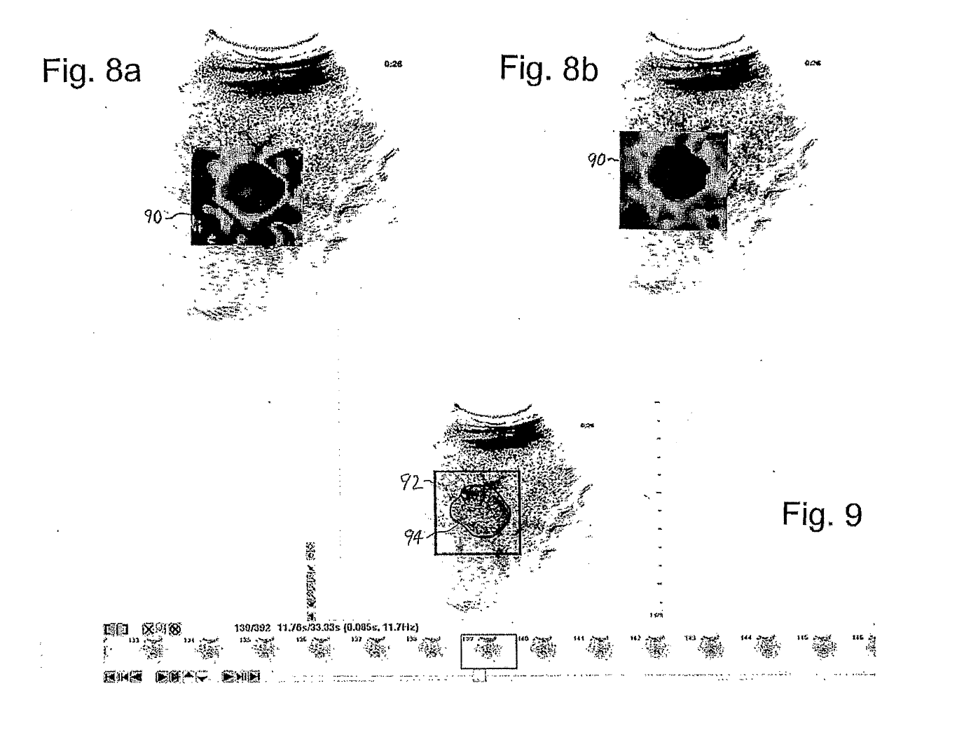

FIGS. 8a and 8b illustrate wash-in period and enhancement period parametric images of a lesion which identify the location of a lesion in a liver image.

FIG. 9 illustrates a border tracing of a lesion using the contrast parametric images of FIGS. 8a and 8b.

Referring first to FIG. 1, an ultrasound system constructed in accordance with the principles of the present invention is shown in block diagram form. An ultrasonic probe 12 includes an array 14 of ultrasonic transducer elements that transmit and receive ultrasonic pulses. The array may be a one dimensional linear or curved array for two dimensional imaging, or may be a two dimensional matrix of transducer elements for electronic beam steering in three dimensions. The array may also be a one dimensional array that is mechanically swept back and forth by the probe to scan a three dimensional volume of the body. The ultrasonic transducers in the array 14 transmit ultrasonic energy and receive echoes returned in response to this transmission. A transmit/receive (“T/R”) switch 22 is coupled to the ultrasonic transducers in the array 14 to selectively couple signals from the transducer elements to A/D converters 30 during the receive phase of operation. The times at which the transducer array is activated to transmit signals may be synchronized to an internal system clock (not shown), or may be synchronized to a bodily function such as the heart cycle, for which a heart cycle waveform is provided by an ECG device 26. When the heartbeat is at the desired phase of its cycle as determined by the waveform provided by ECG device 26, the probe is commanded to acquire an ultrasonic image.

Echoes from the transmitted ultrasonic energy are received by the transducers of the array 14, which generate echo signals that are coupled through the T/R switch 22 and digitized by analog to digital (“A/D”) converters 30 when the system uses a digital beamformer. Analog beamformers may alternatively be used. The A/D converters 30 sample the received echo signals at a sampling frequency controlled by a signal fₛ generated by a central controller 28. The desired sampling rate dictated by sampling theory is at least twice the highest frequency of the received passband, and might be on the order of 30-40 MHz. Sampling rates higher than the minimum requirement are also desirable. Control of the ultrasound system and of various control setting for imaging such as probe selection is effected by user manipulation of the controls of a control panel 20 which is coupled to and applies its control through the central controller 28.

The echo signal samples from the individual transducers of the array 14 are delayed and summed by a beamformer 32 to form coherent echo signals. For 3D imaging with a two dimensional array, it is preferable to partition the beamformer between a microbeamformer located in the probe and the main beamformer in the system mainframe as described in U.S. Pat. No. 6,013,032 (Savord) and U.S. Pat. No. 6,375,617 (Fraser). The digital coherent echo signals are then filtered by a digital filter 34. In this embodiment, the transmit frequency and the receiver frequency are individually controlled so that the beamformer 32 is free to receive a band of frequencies which is different from that of the transmitted band such as a harmonic frequency band. The digital filter 34 bandpass filters the signals, and can also shift the frequency band to a lower or baseband frequency range. The digital filter could be a filter of the type disclosed in U.S. Pat. No. 5,833,613 (Averkiou et al.), for example. Filtered echo signals from tissue are coupled from the digital filter 34 to a B mode processor 36 for B mode processing.

Filtered echo signals of a contrast agent, such as microbubbles, are coupled to a contrast signal processor 38. Contrast agents are often used to more clearly delineate blood vessels, or to perform perfusion studies of the microvasculature of tissue as described in U.S. Pat. No. 6,692,438 (Skyba et al.) for example. The contrast signal processor 38 preferably separates echoes returned from harmonic contrast agents by the pulse inversion technique, in which echoes resulting from the transmission of multiple pulses to an image location are combined to cancel fundamental signal components and enhance harmonic components. A preferred pulse inversion technique is described in U.S. Pat. No. 6,186,950 (Averkiou et al.), for instance.

The filtered echo signals from the digital filter 34 are also coupled to a Doppler processor 40 for Doppler processing to produce velocity and/or power Doppler signals. The output signals from these processors may be scan converted and displayed as planar images, and are also coupled to a 3D image processor 42 for the rendering of three dimensional images, which are stored in a 3D image memory 44. Three dimensional rendering may be performed as described in U.S. Pat. No. 5,720,291 (Schwartz), and in U.S. Pat. Nos. 5,474,073 (Schwartz et al.) and 5,485,842 (Quistgaard), all of which are incorporated herein by reference.

The two dimensional image signals from the contrast signal processor 38, the B mode processor 36 and the Doppler processor 40, and the three dimensional image signals from the 3D image memory 44 are coupled to a Cineloop® memory 48, which stores image data for each of a large number of ultrasonic images. The image data are preferably stored in the Cineloop memory 48 in sets, with each set of image data corresponding to an image obtained at a respective time. The image data in a group can be used to display a parametric image showing tissue perfusion at a respective time during the heartbeat. The groups of image data stored in the Cineloop memory 48 may also be stored in a permanent memory device such as a disk drive or digital video recorder for later analysis. In this embodiment the images are also coupled to a QLAB processor 50, where the images are analyzed and measurements made of characteristics of the images. The QLAB processor is a software package that is commercially available with Philips Healthcare ultrasound systems for various image analysis and quantification procedures. The QLAB processor can be used to make quantified measurements of various aspects of the anatomy in the image such as the delineation of tissue boundaries and borders by automated border tracing as described in U.S. patent publication no. 2005-0075567 and PCT publication no. 2005/054898, and as described below. The QLAB processor is controlled through user manipulation of controls such as buttons and a trackball of the control panel 20. The data and images produced by the QLAB processor are displayed on a display 52 where the user may manipulate, annotate and make measurements of the displayed images through operation of the controls of the control panel 20 as described below.

FIG. 2 illustrates a time-intensity perfusion curve 60 of the type described in U.S. Pat. No. 5,633;613 (Averkiou et al.) Such a perfusion curve 60 may be formed of a succession of echo signals acquired from a particular point in the body as a contrast agent arrives at the point at time t₀, rises to a maximum intensity as the amount of contrast builds up, then decreases as the contrast agent washes out of that point of the vasculature. A number of parameters may be derived by fitting the curve 60 to a perfusion curve model as described above, such as the time t₀ when the contrast agent first arrives at the point in the body, the slope s (or [symbol] ) of a line 62 tangential to the curve 60 where the contrast agent rapidly builds up at the point in the body, and the maximum point A of the curve as the build-up of the contrast agent teaches its peak. Thereafter the curve declines and tails off as the contrast agent is washed out of the vasculature at the point in the body and is gradually replaced by blood which contains no contrast agent. A parametric image may then be formed from one or more of the calculated curve parameters. For instance, an image of the anatomy can be formed with the maximum A value shown at every point in the image. The A values can be represented in a color of a range of colors aligned with the range of A values calculated for all curves. Likewise, a parametric image can be formed with colors depicting the different [symbol] values of the curves at the points in the image, or of a combination of parameters such as (1- [symbol] ) or A/ [symbol] .

FIG. 3 illustrates a method for creating a temporal contrast parametric image in accordance with the present invention. The first step 70 is to acquire ultrasound image data as the contrast agent washes into and out of the region of the body being examined. The contrast agent can be injected into the body of the patient as a bolus of the agent, which is then carried through the blood stream to eventually arrive a number of seconds later at the tissue being imaged. Alternatively a bolus of agent can be formed from a continuous stream of contrast agent by breaking up the continuous stream periodically with higher intensity ultrasound so that the stream has a clear beginning and end as described in U.S. Pat. No. 5,944,666 (Hossack at al.) Images are acquired as the contrast agent washes into and out of the region of the body being studied so that all of the points in the suspect area are rapidly sampled for the presence of contrast agent. The acquired data is stored for analysis. The image data is reviewed to identify a region of interest (ROI) for analysis as step 72. This may be done by locating or drawing a graphic around an ROI as shown by box 82 in the ultrasound image of FIG. 4. The sequences of signals for the points in the ROI are then used in a curve-fitting operation to compute time-intensity curves for the points of the ROI as stated in step 74. In accordance with the principles of the present invention, time-intensity curve levels are set as indicated in step 76 which define three successive periods of time, a wash-in period as the contrast agent builds up, an enhancement period as a maximal level of contrast agent is sustained at each point, and a wash-out period as the contrast agent flows out of the ROI points. These setting may be made in advance of the start of the study or at the beginning of post-processing of the time-intensity curve information. Parametric images may then be formed of each of the time period times as stated in step 78. One or more of the parametric images of the time periods are'then used to delineate a lesion or its boundary in step 80.

FIG. 5 shows an example of time-intensity curve levels which have been set in accordance with step 76 to define time periods for the time-intensity curve 60. In this example the rise or wash-in period is the time duration between a rise of 20% of the peak A of the curve 60, indicated by 63 and time t₁, to a level of 80% of the peak of the curve as indicated by 65 and time t₂. The enhancement period when the amount of contrast agent is around its peak of perfusion is the time duration between the 80% mark of 65 at time t₂ and a decline to 90% of the peak at 67 and time t₃. The fall or wash-out period is the time duration from 90% of the peak at 67 and time t₃ to 30% of the peak at 69 and time t₄. In this example t₁-t₂ is the wash-in period, t₂-t₃ is the enhancement period, and t₃-t₄ is the wash-out period. In the case of a liver tumor the wash-in period occurs during the arterial phase of the heartbeat and the wash-out period occurs during the late portal phase.

Three parametric images may be formed of these time period parameters, one where each image pixel is encoded in accordance with its wash-in time period value, another where each pixel is encoded with its enhancement time period value, and a third where each pixel is encoded with its wash-out time period value. In a constructed embodiment the encoding is done by coloring each pixel with a color from a range of colors corresponding to the range of time period values. Since the values are numeric, the quantification of each point can also be observed. These images and quantifications assist the clinician in diagnosing the lesion being observed. Normal tissue will exhibit a relatively slow wash-in (long rise time period), a slow sustained enhancement (long enhancement time period), and a slow wash-out (long fall time period). Abnormal tissue is characterized by a relatively fast wash-in (short rise time period), a fast enhancement (short enhancement time period), and a fast wash-out (short fall time period). The clinician can observe the time periods in an area of normal tissue outside the lesion and then observe the time periods inside a suspected lesion in the color-coded image, or the quantification of the three time periods at normal and suspect image locations. The comparison will indicate the differences between normal and abnormal tissue.

The clinician can also use the color-coding and quantified values to distinguish between benign and malignant lesions. In the liver, for example, a benign lesion such as FNH (focal nodular hyperplasia) will appear hyper echoic (brighter than surrounding normal tissue) during the arterial phase (rise period), hyper echoic during the enhancement period, and hyper echoic during the portal phase (fall period). A malignant lesion such as HCC (hepatocellular carcinoma) will appear hyper echoic during the arterial phase (rise period), hyper echoic during the enhancement period, and hypo echoic (darker than surrounding normal tissue) during the portal phase (fall period). Additionally, benign lesions tend to have longer enhancement and slower fall time periods than malignant lesions, the latter tending to have shorter enhancement and faster fall time periods than benign lesions. By observing the appearance of the normal tissue background in comparison with the lesion during the time period, an indication of possible malignancy is provided.

One or more of the three time period images may be used to delineate. boundary of a lesion as shown in FIGS. 6 and 7. Boundary delineation is useful in planning and assessing treatment such as radiofrequency ablation or hyperthermic treatment with high intensity ultrasound, for instance. In FIG. 6 the colors of a rise time period image are projected in a three dimensional display 84 with lighter color at a higher projected level and darker colors at a lower projected level. The brighter colors are coded to slow (long) time periods more characteristic of normal tissue while the darker colors are coded for shorter time periods more characteristic of abnormal tissue. The 3D projection may be rotated and turned to assess the extent, degree, and variation of the region of the suspected lesion. Thresholding may then be applied to slice through the projection at selected levels as shown in FIG. 7 to perform region segmentation of areas of the projection. The slice through the 3D projection shown in FIG. 7 illustrates the boundary and the irregular shape of the lesion 82 of this example. Alternatively, a region growing technique (which looks for similarities of homogeneous features) or a border detection technique (which delineates a region by tissue differences) may be used to segment the boundary of the lesion.

FIGS. 8a and 8b each illustrate an ultrasound image of the liver over which is overlaid a color box 90 of a parametric image of a lesion formed in accordance with the present invention. The color box of FIG. 8a contains a rise period parametric image of a region of the liver in the image with a suspected lesion. The color box of FIG. 8b contains an enhancement period parametric image of the same region of the liver. Each parametric image clearly shows the delineation of a lesion with its boundary sharply defined against the normal tissue background of the color box ROI. One or both of the parametric images may be used to draw a line 94 around the border of the lesion in the ROI 92 as shown in FIG. 9. The ROI images may be overlaid and combined by averaging the spatially corresponding pixels, weighting the pixel values differently in the combination, or computing median values of the two images. Thresholding may then be used to define the boundary of the lesion. The lesion boundary may also be found by image processing one or both or a combination of the parametric images. For example a seed point in the interior of the lesion may be indicated and grown to define the area of the lesion. Border-based delineation by identifying discrepancies among neighboring pixels may be used, as may region-based identification techniques which use the homogeneity of the lesion area to classify the pixels of the lesion. The result, as shown in FIG. 9, is a clearly delineated lesion boundary which may be used in planning therapy for the pathology.

What is claimed is:

1. An ultrasonic diagnostic imaging system for identifying a lesion in a region of interest comprising:
a sequence of spatial data sets detecting the rise and fall of an amount of contrast agent which perfuses the region of interest;
a perfusion curve calculator which calculates the time-intensity curve of contrast agent perfusion at spatially different points in the region of interest;
a set of time period delineation values which delineates from each perfusion curve a time period selected from a rise time period, an enhancement time period, and a fall time period;
a parametric image processor which forms a contrast parametric image of the time period values of the selected time period for the region of interest; and
a display which displays the contrast parametric image.

2. The ultrasonic diagnostic imaging system of claim 1, wherein the time period delineation values are levels of a time-intensity curve.

3. The ultrasonic diagnostic imaging system of claim 2, wherein the time period delineation values are defined as percentages of the peak of a time-intensity curve.

4. The ultrasonic diagnostic imaging system of claim 2, wherein the rise time period is a duration during which the amount of contrast agent at a point in the region of interest is increasing, the enhancement time period is a duration during which the amount of contrast agent is at or near its peak, and the fall time period is a duration during which the amount of contrast agent is decreasing.

5. The ultrasonic diagnostic imaging system of claim 4, wherein the rise time period occurs during contrast agent wash-in and the fall time period occurs during contrast agent wash-out.

6. The ultrasonic diagnostic imaging system of claim 1, wherein a parametric image of perfusion phases of relatively long time period values characterizes normal tissue and a parametric image of perfusion phases of relatively shorter time period values characterizes abnormal tissue.

7. The ultrasonic diagnostic imaging system of claim 6, wherein a parametric image with an enhancement perfusion phase of relatively long time period characterizes benign tissue and a parametric image with an enhancement perfusion phase of relatively shorter time period characterizes malignant tissue.

8. The ultrasonic diagnostic imaging system of claim 7, further comprising a contrast signal processor which forms a contrast image of the intensity of contrast agent at different points in the region of interest,
wherein benign tissue is relatively hyper echoic in a contrast image of the fall time period, and malignant tissue is relatively hypo echoic in the contrast image of the fall time period.

9. The ultrasonic diagnostic imaging system of claim 1, further comprising a border detector responsive to the contrast parametric image which delineates the border of a lesion.

10. The ultrasonic diagnostic imaging system of claim 9, wherein the border detector delineates the border of the lesion by thresholding the contrast parametric image.

11. The ultrasonic diagnostic imaging system of claim 9, wherein the parametric image processor is further operable to form a second parametric image of the time period values of a second selected time period,
wherein the first and second parametric images are both used by the border detector to delineate the border of the lesion.

12. The ultrasonic diagnostic imaging system of claim 11, wherein the first and second parametric images are combined by at least one of weighting or averaging.

13. The ultrasonic diagnostic imaging system of claim 9, wherein the border detector utilizes at least one of border-based or region-based pixel processing.

14. A method for identifying abnormal tissue in an ultrasound image comprising:
identifying a region of interest;
acquiring ultrasound data of the region of interest as contrast agent washes in and out of the region of interest;
computing time-intensity curves for points in the region of interest;
identifying at least one of the parameters of a rise time period, an enhancement time period, or a fall time period for each of the time-intensity curves; and
forming a contrast parametric image of at least one of the time period parameters.

15. The method of claim 14 further comprising:
setting levels of a time-intensity curve which define a desired time period of the time-intensity curves.
